(19) United States
(12) Patent Application Publication
(10) Pub. No.: US 2016/0122833 A1
(43) Pub. Date: May 5, 2016

(54) Quasispecies analysis of JC virus DNA present in urine of healthy subjects

(71) Applicant: JANSSEN DIAGNOSTICS BVBA, Beerse (BE)

(72) Inventors: Tom Van Loy, Westerlo (BE); Lieven Jozef Stuyver, Herzele (BE); Luc Remi Mathilde Tritsmans, Vlimmeren (BE)

(21) Appl. No.: 14/784,381

(22) PCT Filed: Apr. 17, 2014
(86) PCT No.: PCT/EP2014/057945

§ 371 Date: Oct. 14, 2015

(30) Foreign Application Priority Data
Apr. 18, 2013 (EP) 13164248.0

(51) Int. Cl. C12Q 1/70

(52) U.S. Cl. CPC C12Q 1/701; C12Q 2600/156

(57) ABSTRACT

JC virus (JCV) is a human polyomavirus that infects the majority of people without apparent symptoms in healthy subjects. A neuropathogenic JCV variant is the causative agent of progressive multifocal leucoencephalopathy (PML), a disorder following lytic infection of oligodendrocytes that mainly manifests itself under immunosuppressive conditions. A hallmark for JCV isolated from PML-brain is the presence of rearrangements in the non-coding control region (NCCR) interspersed between the early and late genes on the viral genome. Such rearrangements are believed to originate from the archetype JC virus variant which is shed in urine by healthy subjects and PML patients. Next generation sequencing (pyro-sequencing) has been performed to explore the NCCR variability in urine of healthy subjects in search for JCV quasispecies and rearrangements reminiscent of PML.

The present invention relates to a method for detecting JC virus quasi species in body fluid.

Human polyoma viruses (HpyVs) are non-enveloped DNA viruses with a ˜5 kilobase pair circular, double-stranded DNA genome. Several members of this virus family are associated with human pathologies. JC virus (JCV) infection can result in a severe clinical outcome i.e. progressive multifocal leucoencephalopathy (PML), an often fatal neurological disorder resulting from a demyelination process after lytic infection of oligodendrocytes. PML establishes itself predominantly in patients suffering from immune deficiencies (e.g. HIV-patients) or patients undergoing immuno-modulatory therapies. For instance, PML-cases have been reported for patients treated with immuno-modulatory monoclonal antibodies such as natalizumab and rituximab. BK virus (BKV), the polyomavirus most closely related to JCV, has been shown to be involved in nephropathy after kidney transplant and may also be an opportunistic pathogen of the central nervous system. More recently, other human polyoma viruses such as Merkel cell polyomavirus (MCV) and TSPyV have also raised interest as oncogenic viruses.

The genome of JC virus encodes both structural and regulatory proteins. Small t-antigen (stAg) and large T-antigen (LTAg) are early expressed regulatory proteins resulting from alternative splicing of the same primary transcript and play a key role in viral replication. Late genes encode the viral capsid proteins VP1, VP2, and VP3, as well as agnoprotein, another regulatory protein. Expression of early and late genes is regulated by a bi-directional non-coding control region (NCCR) which is positioned on the genome between the coding parts of the early and late genes. This control region carries the viral origin of replication (ori) immediately followed by DNA sequence motifs recognized by host transcription factors. As such the NCCR is a key determinant in regulating viral replication and early and late transcription events in host cells permissive for JCV infection, as has been experimentally shown in several in vitro cell studies.

Within an infected host at least two main JC virus variants can exist that differ predominantly in the organization of the NCCR. In the current view, following infection a non-pathogenic form of JC virus can persist in a variety of cells types including, but not limited to, kidney epithelial cells, tonsils and B cell precursors in bone marrow. Primary infection in general occurs without apparent symptoms. Based on the prevalence of anti JCV-antibodies directed against the major capsid protein VP1, it has been estimated that the vast majority of humans have experienced JC virus infection, often already during childhood. A subpopulation of infected people also shed JCV DNA in their urine (viruria). The naturally occurring JCV variant found in urine of both healthy individuals as well as PML-patients is known as the archetype JC virus and is characterized by a typical, well-conserved architecture of the non-coding control region i.e. it is build up of a defined sequence of DNA motifs referred to as domain a to f. JC virus DNA isolated from brain and cerebrospinal fluid (CSF) from patients diagnosed with PML typically carries multiple genomic rearrangements in the NCCR that are believed to have evolved from the archetype virus by deletions and duplications and that can be hyper variable between individual PML-cases.

Since archetype JC virus has not been associated with PML, it might be suggested that rearrangements (i.e. insertions and deletions) as well as single nucleotide changes might be a driving force for viral replication and/or gene transcription in glial cell types ultimately triggering the lytic phase. Hence, the accumulation of DNA variation in the NCCR might alter JCV tropism by changing DNA binding sites for cellular transcription factors in cells permissive for infection. Investigating the naturally occurring variability in the non-coding control region of JC virus and the presence of quasi species seems of particular interest since, upon potential dissemination of the virus within the host, these rearrangements might support viral replication in cell types other than the primary sites of infection.

So there is definitively a need to develop a quick and reliable method for detecting JC Virus quasi species in body fluid of a human being.

A detailed analysis was set up for the presence of genomic rearrangements and nucleotide variability in the non-coding control region of JC virus DNA as it is isolated from urine of healthy subjects. As a gold standard, Sanger sequencing was used to determine the consensus NCCR sequence and the VP1 coding sequence for the examined samples. Next, 454 pyro-sequencing was applied on a subset of 54 samples to study in detail the presence of potential JCV quasispecies i.e. the presence of additional viral variants within a single sample present at a lower frequency than the consensus NCCR sequence. In accordance with the invention evidence has been obtained for the existence of JC virus quasispecies within naturally infected hosts. So it appears surprisingly that JC virus DNA in urine is not always restricted to one unique virus variant, but can be a mixture of naturally occurring variants (quasispecies) reflecting the susceptibility of the NCCR for genomic rearrangements in healthy individuals. This finding could pave the way to explore the presence of JC viral quasispecies and the altered viral tropism that might go along with it as a potential risk factor for opportunistic secondary infections such as PML.

The present invention concerns a method for detecting JC Virus quasi species in body fluid of a human being by:
a) obtaining samples of body fluid such as urine, CSF or blood,
b) extracting DNA from said body fluid,
c) performing a nucleic acid amplification step on said DNA,
d) selecting the JC viral DNA containing samples,
e) performing a further nucleic acid amplification step on said selected JC viral DNA using a primer set comprising JC viral non-coding control region (NCCR) specific primers in order to obtain amplified JC viral non-coding control region (NCCR) DNA,
f) pooling equimolar amounts of said JC viral NCCR DNA and sequencing thereafter said pooled JC viral NCCR DNA via a deep-sequencing technique,
g) aligning the obtained sequences with a reference sequence such as the archetype NCCR sequence and
h) detecting JC virus quasi species in said body fluid.

The current invention preferably relates to a method for detecting JC Virus quasi species in body fluid of a human being by:
a) obtaining samples of body fluid such as urine, CSF or blood,
b) extracting DNA from said body fluid,
c) performing a quantitative PCR amplification on said DNA using a primer set and a probe, wherein the primer set comprises SEQ ID No: 1 (5′ agagtgttgggatcctgtgtttt 3′ and SEQ ID NO: 2 (5′gagaagtgggatgaagacctgttt 3′) and wherein the probe comprises SEQ ID NO: 3 (5′tcatcactggcaaacatttcttcatggc3′),
d) selecting the JC viral DNA containing samples,
e) performing a further PCR amplification on said selected JC viral DNA using a primer set comprising the template specific primers 5′ gattcctccctattcagcactttg 3′ (SEQ ID NO: 4, Fwd primer) and 5′ tccactccaggttttactaa 3′ (SEQ ID NO:5, Rev primer), said primers are attached to the sequence key TCAG and a multiplex identifier sequence (MID) in addition to the primer sequence A (SEQ ID NO: 6: 5′cgtatcgcctccctcgcgcca 3′; Fwd primer) and primer sequence B (SEQ ID NO: 7 5′ctatgcgccttgccagcccgc 3′; Rev primer), in order to obtain amplified JC viral non-coding control region (NCCR) DNA,
f) pooling equimolar amounts of said JC viral NCCR DNA and sequencing thereafter said pooled JC viral NCCR DNA via the pyro-sequencing technique,
g) aligning the obtained sequences with a reference sequence such as the archetype NCCR sequence and
h) detecting JC virus quasi species in said body fluid.

The samples mentioned above can be obtained from any source of body fluid, but are preferably obtained from urine, blood or cerebrospinal fluid (CSF).

In addition, to enrich the viral DNA in the sample of the body fluid, basically any nucleic acid amplification technique may be used, but the most preferred amplification technique is the polymerase chain reaction method (PCR).

The probe used in the quantitative PCR amplification step according to the invention may comprise at its 5′ end a so-called FAM tag while at its 3′end a so-called TAMRA tag is attached.

With FAM and TAMRA is meant a fluorescent reporter (FAM) and a quencher dye (TAM RA).

Other tags known in the art having the same function as the FAM and TAMRA combination can be used accordingly.

With the term “pyro-sequencing” is meant a method of DNA sequencing (determining the order of nucleotides in DNA) based on the “sequencing by synthesis” principle. It differs from Sanger sequencing, in that it relies on the detection of pyrophosphate release on nucleotide incorporation, rather than chain termination with dideoxynucleotides. The technique was developed by Mostafa Ronaghi and Pål Nyrén at the Royal Institute of Technology in Stockholm in 1996. The desired DNA sequence is able to be determined by light emitted upon incorporation of the next complementary nucleotide by the fact that only one out of four of the possible A/T/C/G nucleotides are added and available at a time so that only one letter can be incorporated on the single stranded template (which is the sequence to be determined). The intensity of the light determines if there is more than one of these “letters” in a row. The previous nucleotide letter (one out of four possible dNTP) is degraded before the next nucleotide letter is added for synthesis: allowing for the possible revealing of the next nucleotide(s) via the resulting intensity of light (if the nucleotide added was the next complementary letter in the sequence). This process is repeated with each of the four letters until the DNA sequence of the single stranded template is determined.

Although the so-called 454 sequencing technique (pyro-sequencing) is preferred in the method according to the invention, other next generation sequencing techniques can equally be used in performing the steps of the invention above mentioned.

The term “archetype” refers to a reference sequence and the like for comparing the sequences obtained and to be aligned with.

The term “viral quasi-species” means that more than one viral sequence variant is present in a given sample tested and refers to those sequences not detectable by conventional Sanger sequencing methods known in the art.

In order to describe the current invention, a detailed procedure is disclosed hereafter further clarifying the different steps according to the invention.

The sequences disclosed in the current application are:

| | |
|---|---|
| SEQ ID No: 1 is | 5′ agagtgttgggatcctgtgtttt 3′ |
| SEQ ID NO: 2 is | 5′ gagaagtgggatgaagacctgttt 3′ |
| SEQ ID NO: 3 is | 5′ tcatcactggcaaacatttcttcatgg |
| | c 3′ |
| SEQ ID NO: 4 is | 5′ gattcctccctattcagcactttg 3′ |
| SEQ ID NO: 5 is | 5′ tccactccaggttttactaa 3′ |
| SEQ ID NO: 6 is: | 5′cgtatcgcctccctcgcgcca 3′ |
| SEQ ID NO: 7 is: | 5′ctatgcgccttgccagcccgc 3′ |
| SEQ ID NO: 8 is: | 5′ cctcaatggatgttgccttt 3′ |
| SEQ ID NO: 9 is: | 5′ aaaaccaaagacccctc 3′ |
| SEQ ID NO: 10 is: | 5′ ctattcagcactttgtccattttagc 3′ |
| SEQ ID NO: 11 is: | 5′ ggttttactaactttcacagaagcct 3′ |
| SEQ ID NO: 12 is: | 5′ctttacttttagggttgtacgggac 3′ |
| SEQ ID NO: 13 is: | 5′ tgaggatctaacctgtggaa 3′ |
| SEQ ID NO: 14 is: | 5′ctcccccaaaataactgcaact 3′ |
| SEQ ID NO: 15 is: | 5′ tcctctccactgctggga 3′ |
| SEQ ID NO: 16 is: | 5′ aaggtagggaggagctg 3′ |
| SEQ ID NO: 17 is: | 5′ cccttgtgctaggggtt 3′ |
| SEQ ID NO: 18 is: | 5′ cccttgtgctttgtttactt 3′ |
| SEQ ID NO: 19 is: | 5′ tatgggaggggtttcact 3′ |
| SEQ ID NO: 20 is: | 5′ tatgggaggggcagtg 3′ |

### Healthy Subject Samples

A total of 254 healthy subjects (HSs) were selected for this study: 135 women and 119 men with an age ranging from 19 up to 66 years (median age 42y and average age 42.2y). Urine samples were collected and stored at −80° C. until further processing.

### JC Virus Viral Load Assay

DNA was extracted from 200 μl or 1 ml urine aliquots using the NucliSENS® easyMAG® reagents and platform (Biomérieux). DNA was eluted in 25 μl final volume. The presence of JC virus DNA was determined by quantitative Polymerase Chain Reaction (qPCR) utilizing a primer set (i.e. SEQ ID NO: 1 and SEQ ID NO: 2) and a FAM/TAMRA labeled internal probe (SEQ ID NO: 3) designed to amplify a JC virus large T (LTAg) gene fragment. To quantify the viral load the targeted LTAg gene fragment was subcloned into a pMA backbone (Life Technologies). A 10-fold serial dilution of linearized plasmid DNA was prepared covering a dynamic range of 10 to 10⁸ calculated copy numbers per 5 μl. Another plasmid carrying the homologous BK virus LTAg gene fragment was prepared similarly and included as negative control plasmid. For each sample a 15 μl pre-PCR mixture was prepared containing: 10 μl LightCycler® Probe master (2×) (Roche), 0.06 μl primer SEQ ID NO: 1 (100 μM), 0.06 μl primer SEQ ID NO: 2 (100 μM), 0.04 μl probe SEQ ID NO: 3 (100 μM) and 4.84 μl PCR grade water. 5 μl of DNA extracted from urine, plasmid DNA or PCR grade water (i.e. no template control) was added. Samples were run in duplicate on the BioRad CFX 96 thermocycler with following cycling conditions: 95° C. for 5 min, followed by 40 cycles of 95° C. for 10 s, 60° C. for 10 s and 72° C. for 10 s. qPCR data were analyzed with the BioRad CFX Manager™ software v2.1. and the JC virus VL calculated and expressed as log copies per ml urine.

### Sanger Sequencing of JC Virus Non-Coding Control Region and VP1 Coding Sequence

Viral DNA was extracted from urine as described above and used as template for outer PCR using Phusion high fidelity master mix (2×) (New England Biolabs) and template specific primers: [5′ gattcctccctattcagcactttg 3′ (SEQ ID NO: 4; Fwd primer) and 5′ tccactccaggttttactaa 3′ (SEQ ID NO: 5; Rev primer); JCV NCCR] and [5′ cctcaatggatgttgccttt 3′ (SEQ ID NO: 8; Fwd primer) and 5′ aaaaccaaagacccctc 3′ (SEQ ID NO: 9; Rev primer); JCV VP1 coding sequence]. Cycling conditions for PCR were: 98° C. for 30 seconds followed by 40 cycles of 98° C. for 10 sec, 60° C. for 20 sec and 72° C. for 20 sec and a final step at 72° C. for 5 min. Generated PCR products were subsequently used as template for sequencing PCR using BigDye termination sequencing reagents (Applied Biosystems) and sequencing primers: [5′ ctattcagcactttgtccattttagc 3′ (SEQ ID NO: 10; Fwd primer NCCR) and 5′ ggttttactaactttcacagaagcct 3′ (SEQ ID NO: 11; Rev primer NCCR] or [5′ctttacttttagggttgtacgggac 3′ (SEQ ID NO: 12; Fwd primer1 VP1); 5′ tgaggatctaacctgtggaa 3′ (SEQ ID NO: 13; Fwd primer2 VP1); 5′ctcccccaaaataactgcaact 3′ (SEQ ID NO: 14; Rev primer1 VP1) and 5′ tcctctccactgctggga 3′ (SEQ ID NO: 15; Rev primer2 VP1). Sequencing PCR was run as follows: 96° C. for 1 min followed by 35 cycles of 96° C. for 10 sec, 50° C. for 5 sec and 60° C. for 4 min. Samples were purified [DyeEx 2.0 Spin kit (Qiagen)] and run on the 3730xl DNA Analyzer (Applied Biosystems). DNA sequences were analyzed with the SeqScape v2.5 software.

### Phylogenetic Analysis of HS Samples

Full length VP1 coding sequences (1065 bp) obtained from JCV DNA positive HS samples (n=61) were used for phylogenetic analysis. All sequences were first compared to VP1 coding sequences retrieved from JC virus genotype reference strains in a multiple sequence alignment using clustalW2 (http://www.ebi.ac.uk/Tools/msa/clustalw2/). Data were further processed with the MEGA 5.05 software to generate a phylogenetic tree using Neighbour Joining method. The VP1 coding sequence of JCV genotype 6 was used to root the tree.

### 454 Amplicon Sequencing on GS Junior Platform

### Control Plasmids Included in the Study

An archetype JC virus non-coding control region DNA sequence (CY isolate, NCBI acc. nr. AB038249) was subcloned into the pMA backbone and used as template for PCR amplification. A similar plasmid, but in which a predefined 66 base pairs (bps) deletion corresponding to domain D of the JCV NCCR was introduced, was also generated. XL gold ultracompetent E. coli cells were transformed with both plasmids according to standard procedures and single isolated colonies were cultured overnight at 37° C. in 3 ml LB-medium under selection of 100 μg/ml ampicillin. Plasmid DNA was prepared using the Qiaprep Spin Miniprep kit (Qiagen) following the manufacturer's instructions and used as template in control PCR experiments.

### Amplification of JC Virus Non-Coding Control Region DNA

The “Fusion” primer concept was developed by Roche as part of the amplicon 454 sequencing protocol. The 5′ end of both forward and reverse “Fusion” primers is a 25-mer dictated by the requirements for the 454 sequencing system (primer A, (SEQ ID NO: 6) and primer B, (SEQ ID NO: 7) respectively; see guidelines for amplicon experimental design, Roche) followed by the sequence key “TCAG” and a multiplex identifier sequence (MID) for sample identification. The 3′ portion of the primers consists of target-specific sequences. Here, we designed Fusion primers to amplify a ˜483 bp DNA fragment including the JC virus NCCR. The 3′ end specific sequences were: 5′ gattcctccctattcagcactttg 3′ (SEQ ID NO: 4; Fwd primer) and 5′ tccactccaggttttactaa 3′ (SEQ ID NO: 5; Rev primer). All primers were synthesized at IDT Technologies (Belgium).

PCR was performed on both control plasmid DNA and on viral DNA from urine. All PCRs were run in triplicate and triplicates were pooled afterwards. For each sample a PCR pre-mix was prepared: 10 μl of Phusion high fidelity master mix (2×) (New England Biolabs), 1 μl forward Fusion primer (10 μM), 1 μl reverse Fusion primer (10 μM) and 6 μl of PCR-grade water. 2 μl of plasmid DNA preparation (i.e. ˜100 000 calculated plasmid copies) or 2 μl of extracted viral DNA was used as template (i.e. 20 μl final PCR volume). PCR was run under the following cycling conditions: 98° C. for 30 seconds followed by 40 cycles of 98° C. for 10 sec, 60° C. for 20 sec and 72° C. for 20 sec and a final step at 72° C. for 5 min. DNA amplicons were analyzed by agarose gel electrophoresis (pre stained 1.2% e-gel, Invitrogen), purified with AMPure XP beads (Agencourt) according to the manufacturer's guidelines and eluted in low (10%) TE buffer. DNA was quantified using the Quant-iT picogreen assay kit (Invitrogen). Integrity of the DNA was confirmed on the Bioanalyzer 2100 (Agilent).

### Basic Amplicon Sequencing on Roche GS-Junior Platform and Data Analysis

Equimolar amounts of purified JC virus NCCR amplicons were pooled and sequenced on the GS Junior platform. 454 sequencing was performed at VIB Nucleomics core [http://www.nucleomics.be/, Leuven, Belgium)]. All retrieved DNA sequences were subject to quality assessment. DNA sequences with high quality and covering the full JC virus NCCR were mapped onto the reference sequence NCCR from CY isolate, NCBI acc.nr. AB038249).

### 454 Sequencing: Assay Performance and Criteria Set

To demonstrate the sensitivity and reliability of the used 454 sequencing approach several control samples were included in the study design. Plasmid DNA carrying the archetype JC virus NCCR (NCBI AB038249) was used as template for PCR (˜100 000 calculated input copies) which allowed to evaluate for possible errors that can be introduced during the amplification step as well as 454 sequencing errors. To minimize this error rate all PCRs were performed in triplicate, and triplicates were pooled afterwards. Sensitivity of the assay was examined by spiking in a similar plasmid DNA, but harboring a predefined 66 base pair deletion at different ratios (i.e. 10%, 3%, 1% and 0.3%, respectively) before PCR. A considerable number of non-genuine DNA variations (i.e. deletions, insertions and single nucleotide variations) were present in less than 1% of the analyzed sequences retrieved for the control samples which is generally considered as background variation in 454 sequencing. To clean up the data and to increase reliability of the DNA variations observed, three criteria were set to evaluate potential DNA variations: 1) the background error rate was set at 1% meaning that only DNA variations detected in at least 1% of the viral sequences within a given sample are kept, 2) such variations should be detected by both forward and reverse sequenced DNA molecules at 1% and 3) DNA variations at homopolymeric DNA motifs cannot be reliably interpreted. The control samples in which the predefined 66 bp deletion was spiked in before PCR could be accurately detected down to the level of ˜1% while no other false deletions and/or single nucleotide variations were observed. From the control experiments it was concluded that the used approach was reliable.

### Nested PCR for Validation of Deletions in JC Virus NCCR

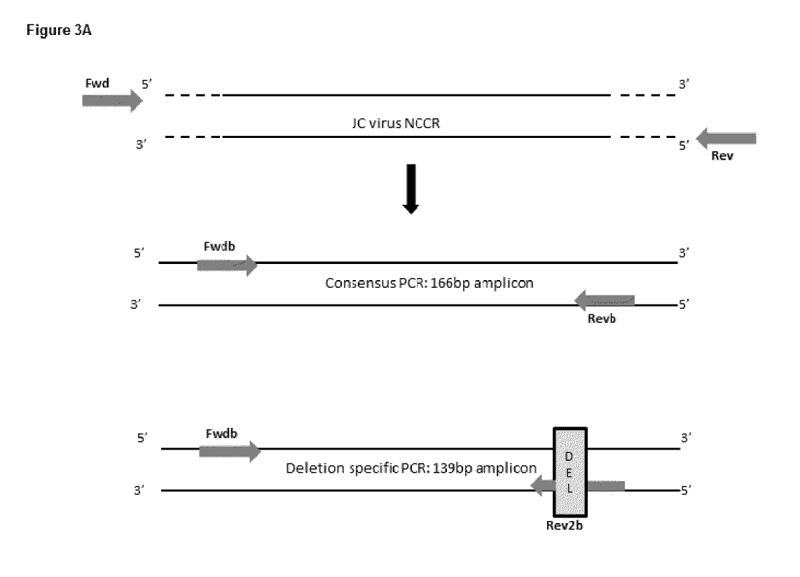

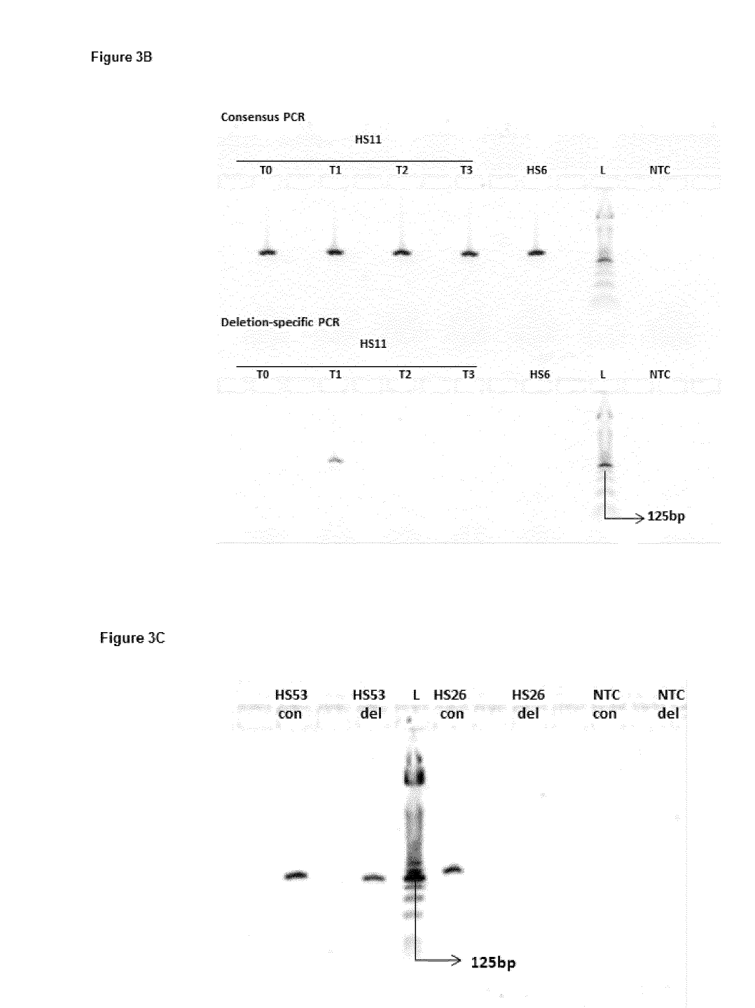

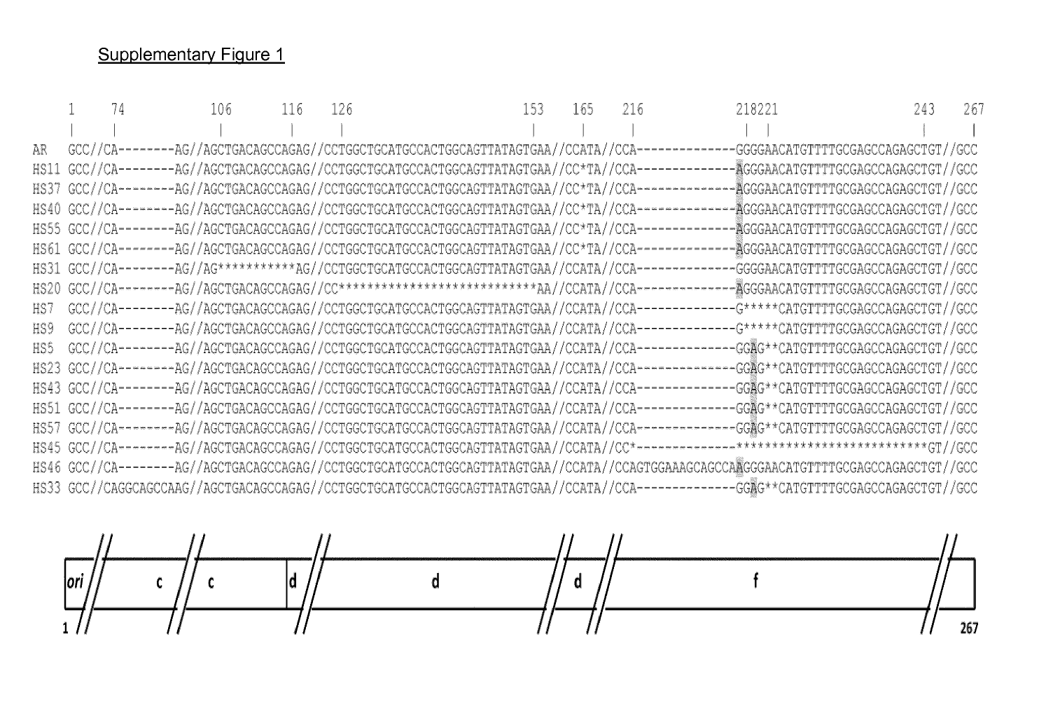

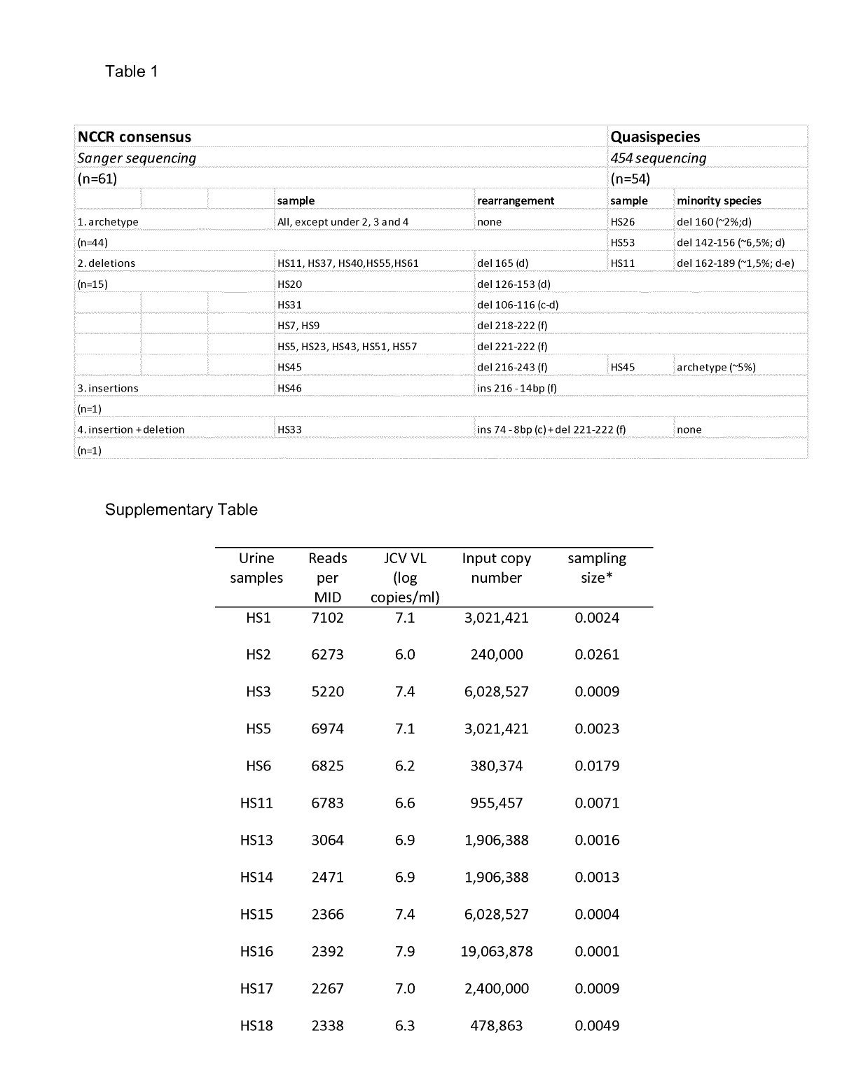

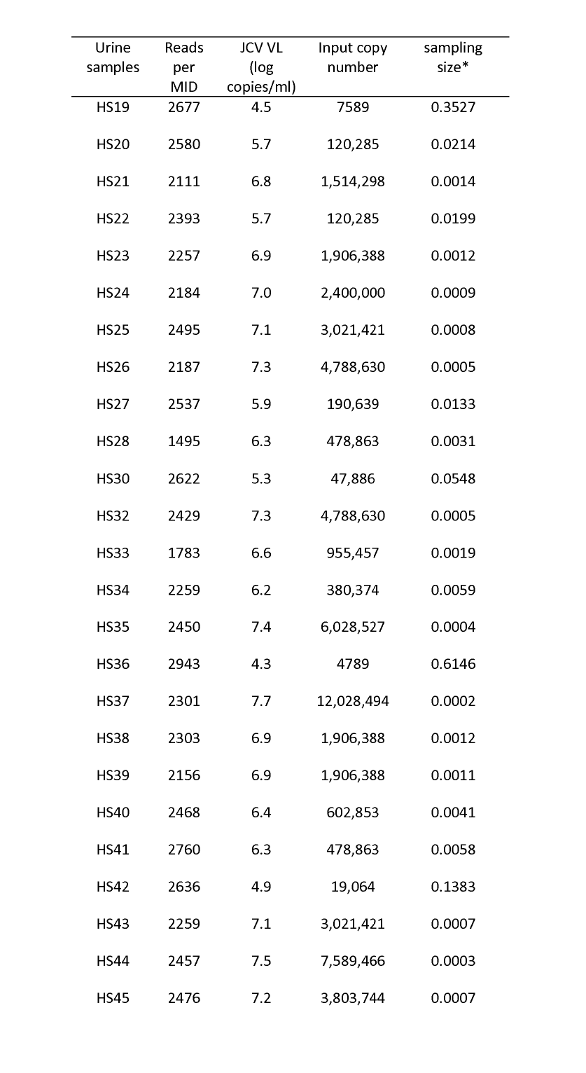

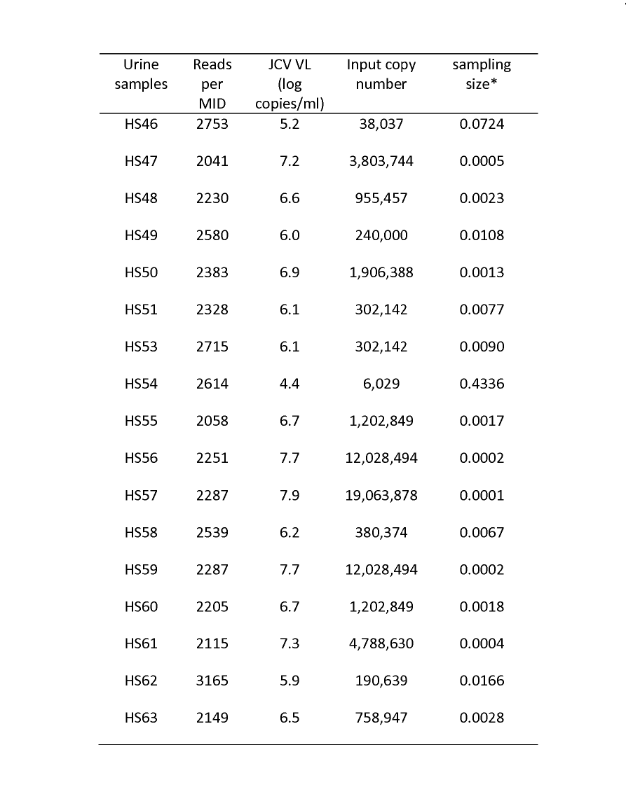

Viral DNA was extracted from urine samples of HS6 and HS11 as described above. Of note, HS11 was a healthy subject that donated several consecutive urine samples within a time interval of ˜1 year [at time point T0 (base line), T1 (˜7 months later), T2 (˜8 months later) and T3 (˜9.5 months later). First, outer PCR was performed on viral DNA using the same primers as for Sanger sequencing. A PCR pre-mix was prepared: 10 μl of Phusion high fidelity master mix (2×) (New England Biolabs), 1 μl forward primer (10 μM), 1 μl reverse primer (10 μM) and 3 μl of PCR-grade water. 5 μl of viral DNA was added as template and PCR was run: 98° C. for 30 seconds followed by 40 cycles of 98° C. for 10 sec, 60° C. for 20 sec and 72° C. for 20 sec and a final step at 72° C. for 5 min. The outer PCR product was diluted 1:100 in distilled water and used as template for two separate inner PCRs: one using the primers Fwdb (5′ aaggtagggaggagctg 3′; SEQ ID NO: 16) and Revb (5′ cccttgtgctaggggtt 3′; SEQ ID NO: 17) and one using the primers Fwdb and Rev2b (5′ cccttgtgctttgtttactt 3′; SEQ ID NO: 18) (see FIG. 3A). Inner PCRs were run as follows: 98° C. for 30 seconds followed by 30 cycles of 98° C. for 10 sec, 60° C. for 15 sec and 72° C. for 15 sec and a final step at 72° C. for 5 min. DNA amplicons were analyzed on pre-stained agarose gels (4% e-gel, Invitrogen).

One μl of viral DNA from urine of HS53 was used in an outer PCR as described above. 1:100 diluted PCR product was used as template for two inner PCRs with primers Fwdb (5′ aaggtagggaggagctg 3′; SEQ ID NO: 16) and Rev (5′ tatgggaggggtttcact 3′; SEQ ID NO: 19) or with primers Fwdb and Rev (5′ tatgggaggggcagtg 3′; SEQ ID NO: 20), respectively. Cycling conditions were: 98° C. for 30 seconds followed by 30 cycles of 98° C. for 10 sec, 70° C. for 15 sec and 72° C. for 15 sec and a final step at 72° C. for 5 min. DNA amplicons were analyzed on pre-stained agarose gels (4% e-gel, Invitrogen).

### Results

### Sanger Sequencing of the JC Virus NCCR and VP1 Coding Sequence

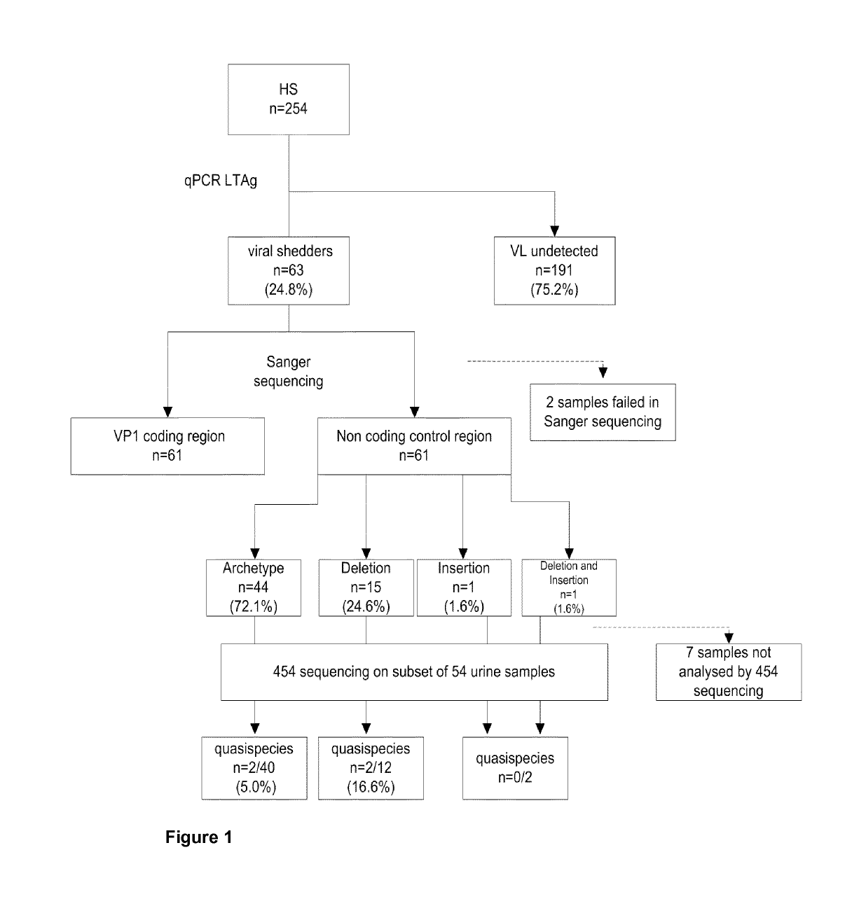

An overview of the experimental set up is given in FIG. 1. Urine samples were donated by 254 HSs. All samples were screened for the presence of JC virus DNA by quantitative PCR and 63 out of 254 HSs (˜24.8%) had shed viral DNA into their urine. A median of 6.60 log copies/ml (range 2.18-7.93) viral load (VL) was determined for these samples. Two μl of extracted viral DNA was used as template for Sanger sequencing of the NCCR. Detailed NCCR sequence information is available in Supplemental FIG. 1. Two samples failed during the procedure likely due to their relatively low VL (2.18 and 2.27 log copies/ml, respectively). In 15 out of 61 samples (˜24.6%) small deletions between 1 and 28 bp were identified in the NCCR while insertions (duplications of 8 and 14 bp, respectively) were present in only two samples (3.3%). One of these duplications was assigned to a sample that also harbored a deletion in the NCCR (HS33, Table 1). Relatively few single nucleotide changes were identified in the NCCR. Only at 7 different positions a single nucleotide variation was present compared to the Japanese CY isolate archetype reference and this variation was mostly restricted to one up to six samples. One exception was at nucleotide position 217 where most samples deviated from the reference sequence (217-A instead of 217-G). This nucleotide change has previously been shown to be a common feature of European and Asian JCV strains.

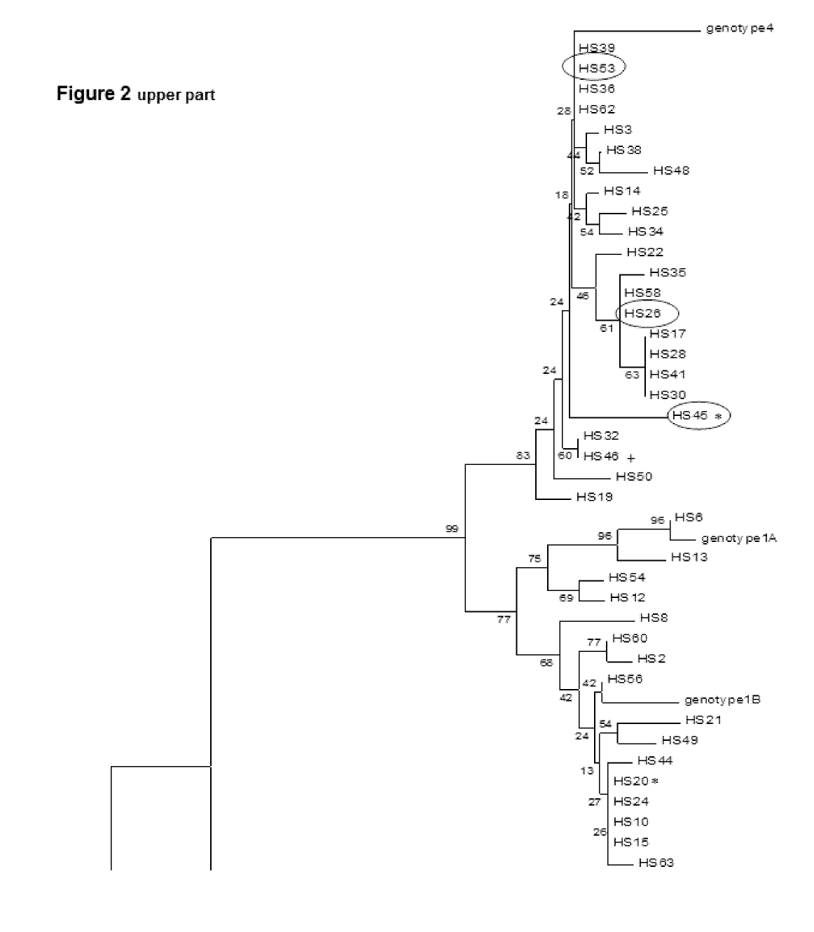

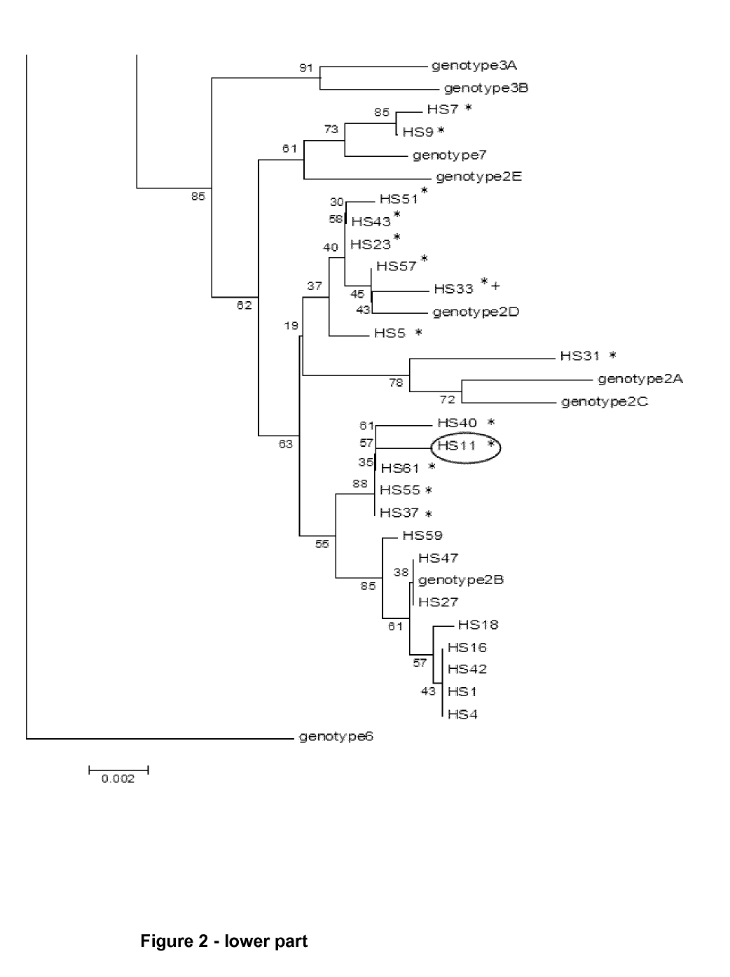

Based on the obtained full length VP1 coding sequences (1065 bp) phylogenetic analysis was performed to assign a JCV genotype to the different samples (FIG. 2). Most of the samples were divided over genotype 1, 2 and 4 while 2 samples clustered together with genotype 7 and no obvious genotype 3 nor genotype 6 samples were present. Eleven out of the fifteen samples carrying a polymorphic deletion in the consensus NCCR were related to genotype 2 JCV while 2 of those samples were genotype 7. The remaining samples were genotype 1 and 4, respectively. Both samples harboring a duplication belonged to genotypes 2 and 4 (FIG. 2).

### Identification of JC Virus NCCR Quasispecies in Urine of Healthy Subjects.

For a total of 54 urine JCV DNA samples an interpretable 454 amplicon sequencing was obtained. Two μl of viral DNA (with variable viral load) was used as template for PCR (performed in triplicate) which implies that at least ˜4789 input copies were used for 454 sequencing (Min: 4789; Max: 19,060,878; Mean: 3,013,724; and Median: 1,358,573; Supplementary Table). A total of 187,420 quality checked DNA sequences covering the full length JCV NCCR were retrieved and divided over 59 samples (i.e. 54 urine samples and 5 plasmid control samples) resulting in an average coverage depth between 1,457 (min.) and 7,044X (max.) per nucleotide position. Due to the relatively high viral load for most of the urine samples, no oversampling (i.e. sequencing the same DNA molecule multiple times) occurred. Hence, no sequencing bias was introduced in the data set (Supplementary Table).

For all samples run in the 454 sequencing protocol the consensus NCCR DNA sequence was obtained after mapping all retrieved DNA sequences against the archetype reference sequence. Importantly, all polymorphic deletions and insertions as they were identified by Sanger sequencing were recovered in these consensus sequences. Beyond the identification of the NCCR consensus sequence, the main goal was to investigate the presence of JCV quasispecies i.e. the presence of minor viral variants within a single sample. Therefore, for each sample all retrieved NCCR sequences deviating from the reference sequence were analyzed under the criteria set for sensitivity and reliability of the assay. Hence, only DNA variations present in at least 1% of the total number of assigned sequences and supported by both forward and reverse sequencing were considered genuine variations. Overall, after analysis of the 454 sequencing data 4 samples out of 54 (7.4%) were identified as containing quasispecies (FIG. 1 and Table 1). Two out of 40 HS without any rearrangements in the consensus NCCR carried deletions present in 2% and 6.5% of the viral sequences, respectively. In addition, 2 out of 12 HS harboring a deletion in the consensus NCCR contained quasispecies while no NCCR quasispecies were identified in both samples carrying a DNA duplication in the consensus non coding control region (Table 1).

Three out of four samples with assigned quasispecies harbored a deletion present in only a minority of sequences. In one of these samples the 1 bp deletion identified by Sanger sequencing (HS11, del 165, Table 1) was part of the larger deletion (del 162-189) identified as minor virus variant in this sample. Of interest, sample HS45 was peculiar since a relatively large 28 bp deletion was identified in 95% of all analyzed sequences after mapping against the reference sequence. Hence, still ˜5% of the NCCR sequences obtained for this sample perfectly matched when compared with the reference archetype sequence.

Next to DNA rearrangements, the intra-sample single nucleotide variability was also investigated by 454 sequencing in search for potential quasispecies characterized by nucleotide replacements. When applying our stringency settings to evaluate the presence of genuine DNA variations single nucleotide replacements could not be identified in any of the samples. Hence, it appeared that no minority viral variants characterized by specific nucleotide replacements were present in the 54 urine samples analyzed by next generation sequencing.

### Validation of JC Virus Quasispecies Identification in Urine of Healthy Subjects

In order to validate the presence of JC virus quasispecies in HS11 (i.e. a 28 bp deletion present in ˜1.5% of the viral DNA sequences, Table 1) a nested PCR approach was followed. Viral DNA was freshly prepared from the following urine samples: HS11 T0 (viral load 7.3 log copies/ml), HS11 T1 (i.e. the urine collection also used for 454 sequencing; viral load 6.6 log copies/m1), HS11 T2 (viral load 7.8 log copies/ml) and HS11 T3 (viral load 7.7 log copies/ml) which gave the opportunity to follow the presence of the detected deletion over time. A nested PCR approach was applied on these samples as described earlier and graphically explained in FIG. 3A. As a negative control, viral DNA extracted from HS6 in which the 28 bps deletion was not detected by 454 sequencing, was included in the assay. As expected a JC virus NCCR DNA fragment was amplified during inner PCR when primers designed to target a consensus fragment, present in both HS6 and HS11, were used (FIG. 3B; upper panel). However, only in sample HS11 T1 a DNA fragment was successfully amplified during inner PCR in which the consensus reverse primer was replaced with a primer over-spanning the potential deletion (FIG. 3B; lower panel). This result confirms that the quasispecies detected in HS11 truly exists and can be specifically attributed to this sample, but in this case is likely to be a transient phenomenon as it was not detected in later samples. Using a similar nested PCR approach the 15 bp deletion identified in ˜6.5% of the DNA sequences from HS53 (Table 1) were also confirmed to be genuine and specific for HS53 (FIG. 3C).

### EXPLANATION OF THE FIGURES

### FIG. 1

### Outline of the Experimental Work Presented.

JC virus DNA was detected in urine of 63 out of 254 healthy subjects (HSs) by qPCR targeting the large T antigen (LTAg) gene. For 61 viral shedders the full length VP1 coding sequence and the non-coding control region (NCCR) DNA was obtained via Sanger sequencing. When compared to a reference archetype NCCR (i.e. CY isolate, NCBI acc. nr. AB038249) 44 samples did not contain any rearrangement while 15 samples (24.6%) harbored a deletion (between 1 and 28 bp), 1 sample (1.6%) carried a duplication in their consensus NCCR sequence and 1 sample carried both an insert and a deletion. A subset of 54 urine JCV DNA samples were further analyzed by next generation sequencing (454 pyro-sequencing) to specifically look at the presence of JCV quasispecies that were identified in 2 out of 40 archetype samples (5.0%) and 2 out of 12 samples (16.6%) that contained a deletion when analyzed by Sanger sequencing. No quasi-species were detected in both samples carrying a duplication in the consensus NCCR.

### FIG. 2

Phylogenetic Analysis Based on the VP1 Coding Sequence from Healthy Subjects.

The full length VP1 coding sequence (1065 bp) was obtained for all healthy subjects for which also the non coding control region (NCCR) was Sanger sequenced (n=61). The relatedness of the HS samples to defined JC virus genotypes is illustrated in a phylogenetic tree. For each JCV genotype the following reference VP1 coding sequences were used (NCBI accession number between brackets): genotype 1A (AF015526), genotype 1B (AF015527), genotype 2A (AF015529), genotype 2B (AF015533), genotype 2C (AF015535), genotype 2D (AF015536), genotype 2E (AF281606), genotype 3A (U73500), genotype 3B (U73501), genotype 4 (AF015528), genotype 6 (AF015537), genotype 7 (U61771). Healthy subjects (HS) in which deletions or insertions were identified in the non coding control region via Sanger sequencing are indicated by * (deletion) or + (insertion). HSs in which JCV quasispecies were identified by 454 sequencing are circled.

### FIG. 3

### Validation of JC Virus Quasispecies by Nested PCR.

(A) A nested PCR approach was developed to validate the presence of the 28 bps deletion identified in ˜1.5% of the viral DNA sequences in HS11.
(B) The nested PCR approach was applied on viral DNA extracted from urine from HS11. Urine aliquots donated at different time points were included: T0, T1, T2 and T3. Viral DNA extracted from HS6 T1 was included as a negative control, together with a no template control (NTC). The upper panel shows successful amplification of the consensus DNA fragment in all samples. In contrast, only in HS11 T1 a PCR fragment could be amplified when using the reverse primer spanning the deletion instead of the consensus reverse primer, confirming the presence of this deletion. L=25 base pair DNA ladder (Invitrogen).
(C) A similar nested PCR approach was used to demonstrate the existence of quasispecies in HS53. DNA fragments were generated when a primer set designed to amplify a consensus sequence from both HS53 and HS26 (lanes HS53 con and HS26 con). When the reverse primer was replaced by a primer specifically targeting the sequence deleted in the quasispecies successful amplification was only detected in HS53 (HS53 del), but not in HS26 (HS26 del). NTC: no template control. L=25 base pair DNA ladder (Invitrogen).

### Table 1

Overview of NCCR rearrangements (i.e. deletions and insertions) identified by Sanger sequencing and 454 sequencing.

Comparison of the consensus NCCR as determined by Sanger sequencing (n=61) revealed four distinct groups of samples: 1) archetype sequences with no DNA rearrangements compared to the reference NCCR (CY isolate), 2) samples harboring polymorphic deletions (del), 3) samples carrying polymorphic insertions (ins) and 4) sample harboring a deletion with an insertion. 454 sequencing was further applied (n=54) to identify JCV quasispecies. Healthy subject (HS) samples in which quasispecies were identified, the nature of the minority sequences contributing to the quasispecies and the percentage of viral NCCR sequences harboring the minority sequences are indicated. Of note, HS11 carries a 1 bp deletion at position 165 that in ˜1.5% of the viral population is part of a more extensive deletion. The sequence blocks (a to f) affected by the NCCR rearrangements are indicated between brackets.

### Supplementary Table

Overview of urine samples from healthy subjects (HSs) used for 454 sequencing of the JC virus non coding control region (NCCR). All samples were assigned a multiplex identifier (MID) for analysis purposes. In total 187,420 DNA sequences (reads) covering the full length JCV NCCR were retrieved and mapped on the reference sequence (i.e. JCV NCCR archetype sequence, CY isolate, from NCBI accession number AB038249). The number of analyzed reads per urine sample is indicated. The JC virus viral load (JCV VL) as determined for the urine samples is expressed as log copies/ml. Based on this VL a sampling size i.e. number of analyzed reads divided by the input copy number was calculated.

### Supplementary FIG. 1

Sequence alignment of the non-coding control region DNA sequence retrieved by Sanger sequencing from viral DNA isolated from urine (n=17). Only samples in which DNA rearrangements (deletions or insertions) were identified in comparison to the archetype reference sequence are included in the alignment. DNA regions in which no rearrangements were present are not shown and are symbolized by −//−. On top of the alignment the archetype NCCR from CY isolate (267 nucleotides, NCBI acc. nr. AB038249) is presented. Deletions are indicated by *. Gaps (−) were introduced in the alignment for proper alignment of the sequences. Single nucleotide changes (compared to the reference sequence) are shaded in grey. Nucleotide numbering of the NCCR is indicated on top of the alignment. The lower bar gives a schematic representation of the NCCR DNA architecture showing in which predefined NCCR domain the identified rearrangements were present. Ori: origin of replication.

1. A method for detecting JC Virus quasi species in body fluid of a human being by:
a) obtaining samples of body fluid such as urine, CSF or blood,
b) extracting DNA from said body fluid,
c) performing a nucleic acid amplification step on said DNA,
d) selecting the JC viral DNA containing samples,
e) performing a further nucleic acid amplification step on said selected JC viral DNA using a primer set comprising JC viral non-coding control region (NCCR) specific primers in order to obtain amplified JC viral non-coding control region (NCCR) DNA,
f) pooling equimolar amounts of said JC viral NCCR DNA and sequencing thereafter said pooled JC viral NCCR DNA via a deep-sequencing technique,
g) aligning the obtained sequences with a reference sequence such as the archetype NCCR sequence and
h) detecting JC virus quasi species in said body fluid.

2. Method according to claim 1 for detecting JC Virus quasi species in body fluid of a human being by:
a) obtaining samples of body fluid such as urine, CSF or blood,
b) extracting DNA from said body fluid,
c) performing a quantitative PCR amplification on said DNA using a primer set and a probe, wherein the primer set comprises SEQ ID No: 1 (5′ agagtgttgggatcctgtgtttt 3′ and SEQ ID NO: 2 (5′ gagaagtgggatgaagacctgttt 3′) and wherein the probe comprises SEQ ID NO: 3 (5′tcatcactggcaaacatttcttcatggc3′),
d) selecting the JC viral DNA containing samples,
e) performing a further PCR amplification on said selected JC viral DNA using a primer set comprising the template specific primers 5′ gattcctccctattcagcactttg 3′ (SEQ ID NO: 4, Fwd primer) and 5′ tccactccaggttttactaa 3′ (SEQ ID NO:5, Rev primer), said primers are attached to the sequence key TCAG and a multiplex identifier sequence (MID) in addition to the primer sequence A (SEQ ID NO: 6: 5′cgtatcgcctccctcgcgcca 3′; Fwd primer) and primer sequence B (SEQ ID NO: 7 5′ctatgcgccttgccagcccgc 3′; Rev primer), in order to obtain amplified JC viral non-coding control region (NCCR) DNA,
f) pooling equimolar amounts of said JC viral NCCR DNA and sequencing thereafter said pooled JC viral NCCR DNA via the pyro-sequencing technique,
g) aligning the obtained sequences with a reference sequence such as the archetype NCCR sequence and
h) detecting JC virus quasi species in said body fluid.


SEQUENCE LISTING (28 sequences)

SEQ ID NO: 1 - DNA, 23 bp, JC polyomavirus
agagtgttgggatcctgtgtttt

SEQ ID NO: 2 - DNA, 24 bp, JC polyomavirus
gagaagtgggatgaagacctgttt

SEQ ID NO: 3 - DNA, 28 bp, JC polyomavirus
tcatcactggcaaacatttcttcatggc

SEQ ID NO: 4 - DNA, 24 bp, JC polyomavirus
gattcctccctattcagcactttg

SEQ ID NO: 5 - DNA, 20 bp, JC polyomavirus
tccactccaggttttactaa

SEQ ID NO: 6 - DNA, 21 bp, JC polyomavirus
cgtatcgcctccctcgcgcca

SEQ ID NO: 7 - DNA, 21 bp, JC polyomavirus
ctatgcgccttgccagcccgc

SEQ ID NO: 8 - DNA, 20 bp, JC polyomavirus
cctcaatggatgttgccttt

SEQ ID NO: 9 - DNA, 17 bp, JC polyomavirus
aaaaccaaagacccctc

SEQ ID NO: 10 - DNA, 26 bp, JC polyomavirus
ctattcagcactttgtccattttagc

SEQ ID NO: 11 - DNA, 26 bp, JC polyomavirus
ggttttactaactttcacagaagcct

SEQ ID NO: 12 - DNA, 25 bp, JC polyomavirus
ctttacttttagggttgtacgggac

SEQ ID NO: 13 - DNA, 20 bp, JC polyomavirus
tgaggatctaacctgtggaa

SEQ ID NO: 14 - DNA, 22 bp, JC polyomavirus
ctcccccaaaataactgcaact

SEQ ID NO: 15 - DNA, 18 bp, JC polyomavirus
tcctctccactgctggga

SEQ ID NO: 16 - DNA, 17 bp, JC polyomavirus
aaggtagggaggagctg

SEQ ID NO: 17 - DNA, 17 bp, JC polyomavirus
cccttgtgctaggggtt

SEQ ID NO: 18 - DNA, 20 bp, JC polyomavirus
cccttgtgctttgtttactt

SEQ ID NO: 19 - DNA, 18 bp, JC polyomavirus
tatgggaggggtttcact

SEQ ID NO: 20 - DNA, 16 bp, JC polyomavirus
tatgggaggggcagtg

SEQ ID NO: 21 - DNA, 15 bp, JC polyomavirus
agctgacagccagag

SEQ ID NO: 22 - DNA, 32 bp, JC polyomavirus
cctggctgcatgccactggcagttatagtgaa

SEQ ID NO: 23 - DNA, 32 bp, JC polyomavirus
ccaggggaacatgttttgcgagccagagctgt

SEQ ID NO: 24 - DNA, 32 bp, JC polyomavirus
ccaagggaacatgttttgcgagccagagctgt

SEQ ID NO: 25 - DNA, 27 bp, JC polyomavirus
ccagcatgttttgcgagccagagctgt

SEQ ID NO: 26 - DNA, 30 bp, JC polyomavirus
ccaggagcatgttttgcgagccagagctgt

SEQ ID NO: 27 - DNA, 46 bp, JC polyomavirus
ccagtggaaagcagccaagggaacatgttttgcgagccagagctgt

SEQ ID NO: 28 - DNA, 12 bp, JC polyomavirus
caggcagccaag